(19) United States
(12) Patent Application Publication
(10) Pub. No.: US 2003/0233030 A1
(43) Pub. Date: Dec. 18, 2003

(54) System for repetitive interval clinical evaluations

(76) Inventor: William H. Rice, Austin, TX (US)

Correspondence Address: Brobeck Phleger & Harrison LLP, 12390 El Camino Real, San Diego, CA, 92130, US

(21) Appl. No.: 10/174,498

(22) Filed: Jun. 17, 2002

(51) Int. Cl. A61B 5/00

(52) U.S. Cl. 600/300

(57) ABSTRACT

A healthcare tool allows a patient to record daily parameters associated with the patient's clinical status, for example, body weight for congestive heart failure patients. A graph may be created showing the parameters on a control chart. The parameters are statistically analyzed against a control range, and when a parameter moves out of the control range, the system automatically creates a pop-up window alerting the patient that the parameter is outside the control range, and that the patient should consider informing a healthcare professional.

### BACKGROUND OF THE INVENTION

1. Field of the Invention

The present invention relates to healthcare systems, and more particularly, to a system for optimization of chronic disease care.

2. Background Art

More than 90 million Americans live with chronic diseases, which account for more than 60% of the nation's medical care costs. By definition, a chronic disease progresses over time and has a generally predictable set of costly exacerbations, complications and recurrences.

There have been significant efforts to optimize cost and quality of the healthcare system. These efforts have been focussed on the development of “best practices” protocols, medical error reduction, bulk purchasing and pharmaceutical benefits management, new medicine, minimally invasive surgery, and redesign of care systems to more effectively manage demand for health services. While each effort is important, none of these addresses the best way to positively impact cost and quality in healthcare (optimization)—early identification of chronic disease exacerbation in a specific patient.

Attempts have been made to utilize computers for providing medical treatment. Computerized schemes have been proposed for communicating automatically with a patient regarding a previously diagnosed disease and optimizing or adjusting a therapy for that disease automatically, using information received from the patient. In the past few years, automated schemes of medical treatment typically involve the use of computers and the internet to treat patients remotely, that is, to allow a doctor to be virtually “present” at the patient's location to help treat the patient at that location. The purpose of these conventional schemes of remote treatment by using computers or the internet is to avoid unnecessary office visits, thereby effecting savings in overall healthcare costs.

However, it has not been possible to identify specific chronic disease patients with evolving complications, exacerbations or recurrences, and thus it has not been possible to provide any early intervention to mitigate the worsening clinical scenario for individual patients. Furthermore, the worsening of a patient's condition from chronic diseases may not be given to predictive models, and is therefore difficult or impossible to treat remotely using a standardized therapy based upon broad demographic models. Therefore, there is a need for a system to allow early detection of chronic disease exacerbations or complications to decrease the need for emergency medical services while measurably improving patient outcomes.

### SUMMARY OF THE INVENTION

The present invention provides a health parameter statistical control measurement tool for optimization of chronic disease care. The system employs a non-linear model that dictates the use of repetitive interval clinical evaluations as a primary tool for the earliest possible detection of a worsening clinical condition in a patient having a specific chronic disease.

A user or patient performs repetitive, interval, clinical evaluations and inputs parameters associated with the patient's chronic disease. The parameters are then compared to the patient's own, previously recorded data. If the parameter falls outside a predetermined statistical control range, the patient is alerted so that the patient follows-up with appropriate healthcare team members.

Advantageously, the system of the present invention allows for early detection of chronic disease care exacerbations, which can decrease the need for services while measurably improving clinical outcomes for the most common diagnoses of chronic disease patients.

### BRIEF DESCRIPTION OF THE DRAWINGS

The present invention will be described with particular embodiments thereof, and references will be made to the drawings in which:

FIG. 1 is a flow diagram illustrating the system according to an embodiment of the present invention;

FIG. 2 is a set up process according to an embodiment of the present invention;

FIGS. 3-6 and 7A-B are exemplary screen shots of the steps performed by the health parameter statistical control measurement tool according to an embodiment of the present invention;

FIG. 8 is an exemplary screen shot of a “Report” according to an embodiment of the present invention;

FIG. 9 is an exemplary screen shot of an additional alerting step according to an embodiment of the present invention;

FIG. 10 is an exemplary screen shot of an “EXIT” step according to an embodiment of the present invention; and

FIG. 11 illustrates a typical computer system.

### DETAILED DESCRIPTION

The present invention relates to a system for optimization of chronic disease care. Chronic disease care optimization herein is defined as the process of early identification of exacerbations, complications and recurrences. This early identification presents the opportunity for specific patients to alert their healthcare provider team and to receive mitigating treatments earlier, and to avoid costly and painful medical interventions and hospitalizations.

A non-linear model of chronic disease indicates that the primary opportunity to optimize chronic disease care is prevention by using repetitive, interval, clinical evaluations.

Primary prevention is defined as efforts to prevent disease processes from occurring. For example, lung cancer, emphysema and cardiac disease may be minimized through efforts to stop smoking. Adult-onset diabetes may be minimized through diet.

Secondary prevention is defined as the effort to decrease the progression of a disease after a patient is diagnosed. For example, people with diabetes may progress and have vision decrements, foot ulcers and renal failure. People with asthma may have frequent emergency department visits and limitations of activity. Secondary prevention efforts seek to minimize the progression and frequency of these exacerbations and complications.

Unlike efforts to address primary prevention, there is essentially no large effort to provide secondary prevention for chronic disease patients. Given the inevitability of chronic disease in an aging population, secondary prevention is the most important effort available for patients.

The non-linear model in this invention is a chaotic model. In a chaotic model, there is a sensitive dependence on initial conditions. Mathematically, the initial conditions of a system, when varied by an exceedingly small amount, result in widely variable outcomes without a distinguishable pattern.

In a chaotic or complex system, there is one way to optimize data, that is, by repetitive measurements. Weather prediction is a classic example of a non-linear system with a “chaotic” component. The National Oceanographic and Atmospheric Association (NOAA) is a component of the Department of Commerce that has a primary mission of gathering data and predicting the weather. Several decades ago, as mainframe computers became available to solve large data set problems, programs to model weather systems began to be written with the purpose of improving NOAA's ability to predict the weather. It was soon discovered that if the input data of the program varied by some exceedingly small amount, for example, if barometric pressure at some location, at some time, increased by an un-measurable thousandth of an inch, then the output of the model was drastically different. Prediction of sunshine changed to prediction of thunderstorms.

To optimize this non-linear system with a “chaotic” component, NOAA has employed a repetitive data sampling system where the critical element is the periodicity of the data sampling. The NOAA system provides the best possible weather predictions only with frequent measurements over time. More intensive measurements taken less frequently are not a reliable approach to optimizing weather prediction.

The idea of employing a repetitive data sampling system has direct application to healthcare. The present invention will be described in relation to a particular application in congestive heart failure (CHF). It should be noted that the present invention is not limited to applications in CHF, but may also be used in applications to other chronic diseases such as diabetes, asthma, emphysema, cancer, and other cardiovascular diseases such as arrhythmia.

CHF is the largest disease class and the most common reason for hospitalization for people over 65 in the United States. In an example, two CHF patients could “look” clinically identical having the exact same medical histories, lifestyle, and clinical findings and can be seen, diagnosed and treated at the exact same time. However, like next year's weather forecast, it cannot be accurately predicted which patient will progress with an uneventful clinical course and which patient will deteriorate and need intensive care.

In this example, the two “identical” patients are analogous to the weather system model, with two seemingly identical initial conditions (differing barometric pressure by an un-measurable thousandth of an inch). Without repetitive monitoring, the weather model cannot predict weather reliably. With repetitive monitoring, however, accurate weather prediction can be obtained for three to ten days.

Interestingly, there is an easily measured parameter that enables prediction of likely exacerbations and complications in CHF patients: body weight. CHF patients have occasional exacerbations that require hospitalization and intensive care. However, there is almost always a predictable sequence of symptoms and findings prior to the patient's “decompensation.” CHF patients often begin a pattern of weight gain. It is this progression of an easily measured parameter that provides the prevention window of opportunity in CHF patients. Mitigation of disease exacerbations consists primarily of alerting the patient, and eventually, the healthcare provider team. When the healthcare team knows that a CHF patient is gaining weight, incremental doses of diuretics and other measures can very effectively prevent the acute clinical exacerbation.

Thus, in chronic disease care, more frequent data inputs can result in early detection of clinical exacerbations and complications. This is the opportunity for secondary prevention to identify an evolving problem before it incapacitates the patient and requires intensive medical intervention.

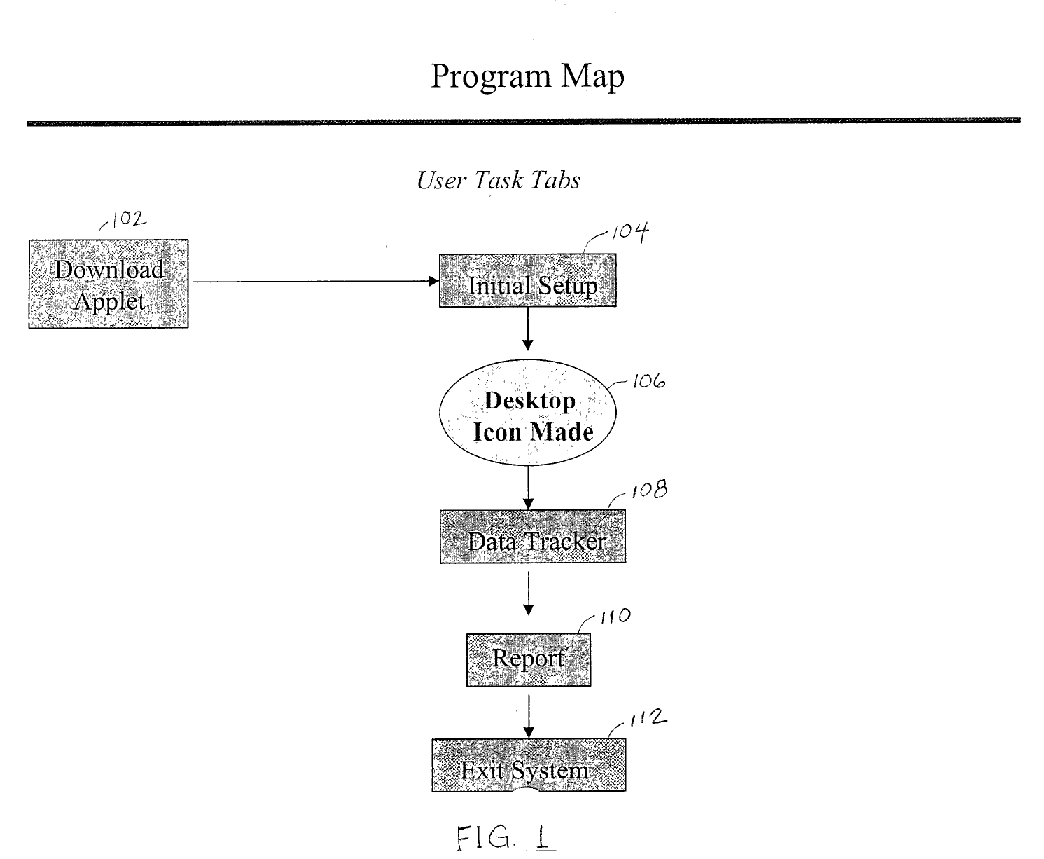

Referring first to FIG. 1, a flow diagram illustrating the system according to an embodiment of the present invention is presented. In step 102, the process starts by downloading a program application, for example, a JAVA applet. Small JAVA applications are called JAVA applets and can be downloaded from a Web server and run on a user's computer by a JAVA-compatible Web browser, such as Netscape Navigator or Microsoft Internet Explorer.

It should be noted that if a second user wants to also use the system, the program application can be written to accommodate additional users or the second user may download the applet another time so that the information is kept separate.

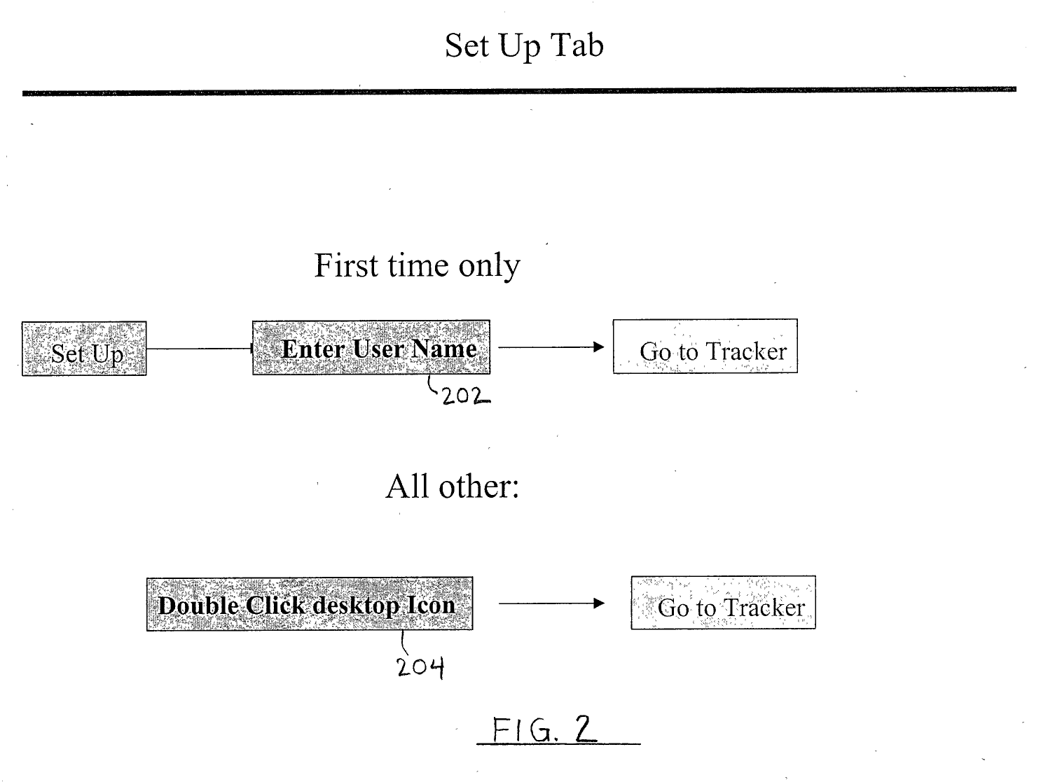

In step 104, after the JAVA applet is downloaded, the user initially sets up the system. In step 106, the process creates a desktop icon. In particular, FIG. 2 illustrates the set up process according to an embodiment of the present invention. In step 202, a first time user inputs an identifying name. The process then continues to the health parameter statistical control measurement tool. A repeat user, in step 204, simply double clicks on a desktop icon to enter the program, and then the process goes to the health parameter statistical control measurement tool.

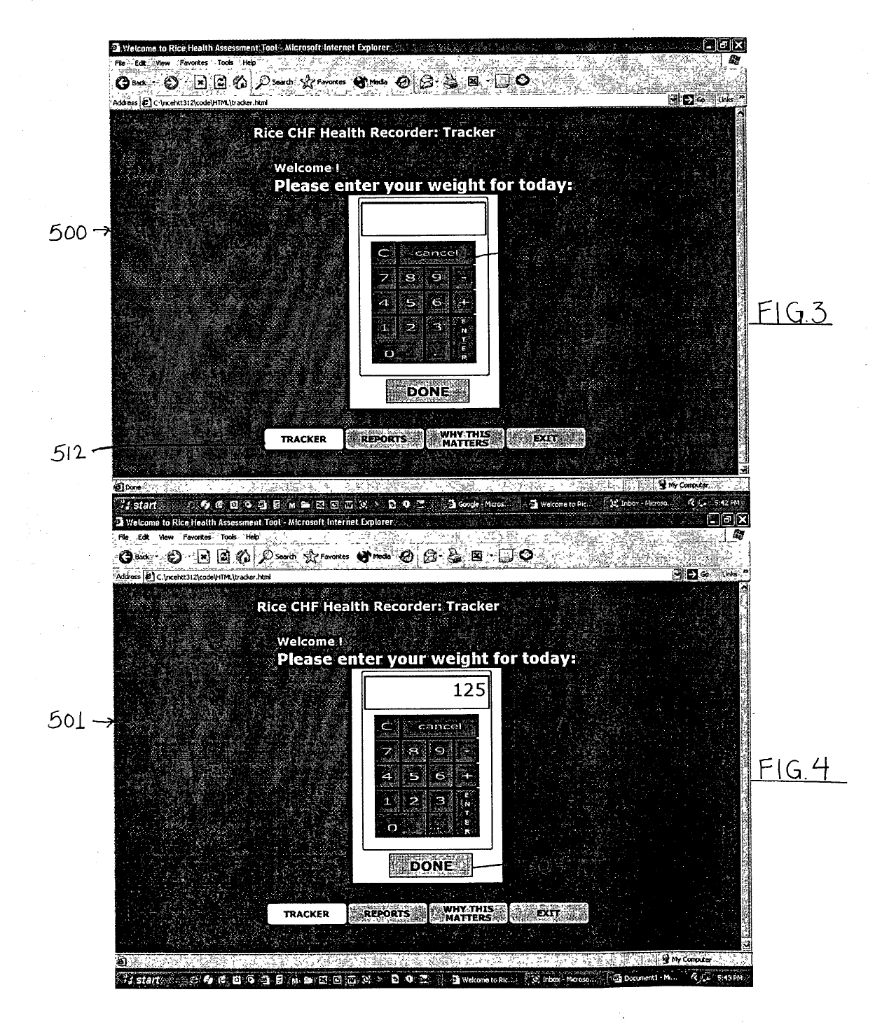

Referring back to FIG. 1, in step 108, the process goes to the health parameter statistical control measurement tool. The health parameter statistical control measurement tool receives inputs or parameters associated with a particular patient's health condition or clinical status. The health parameter statistical control measurement tool will be described in more detail below with respect to FIGS. 3-7.

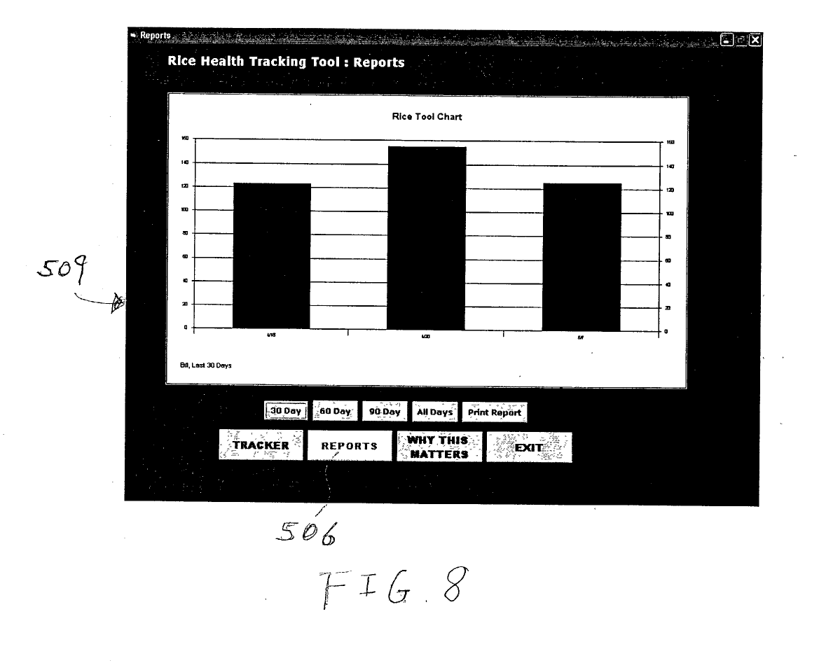

In step 110, a report is generated, which may comprise a graph covering a desired time frame selected by the user. In step 112, the process exits the system. These steps will be explained in more detail below with respect to FIGS. 8-10.

Referring now to FIGS. 3-7, exemplary screen shots of the steps performed by the health parameter statistical control measurement tool according to an embodiment of the present invention are presented. After a user signs into the system, the system goes to the health parameter statistical control measurement tool as indicated by the highlighted button “TRACKER” 512 of screen shot 500 of FIG. 3.

As discussed above, the system will be described in conjunction with an application for a CHF patient wherein the parameter of body weight is tracked to prevent exacerbations of the patient's condition. Because many other chronic diseases have easily measured parameters highly associated with the patient's clinical status, the system of this invention can be broadly applied to the care of these diseases as well. Common as well as expensive chronic diseases in the United States that may be tracked include: asthma, for which peak flow can be measured; chronic obstructive pulmonary disease (emphysema), for which peak flow can be measured; diabetes, for which glucose can be measured; other cardiovascular diseases such as arrhythmia, infarction, ischemia, atherosclerosis, etc., for which number of nitro tablets taken daily, number of chest pain episodes, ambulation distance without pain, minutes walking without pain, etc. can be measured; rehabilitation, such as from hip and knee replacements, for which ambulation paces/activity can be measured; cancer, post chemotherapy/post radiation, for which symptoms of toxicity such as number of emesis, diarrhea, food/liquid intake, etc. can be measured.

A CHF patient is prompted to enter his or her measured body weight by clicking on number pad 502 as illustrated in screen shot 500. FIG. 4 illustrates an example in which the patient entered a weight of 125. Once the patient enters the weight, the button 504 labeled “Done” may be pressed to continue. It should be noted that in other embodiments, the patient may be asked to confirm the entry.

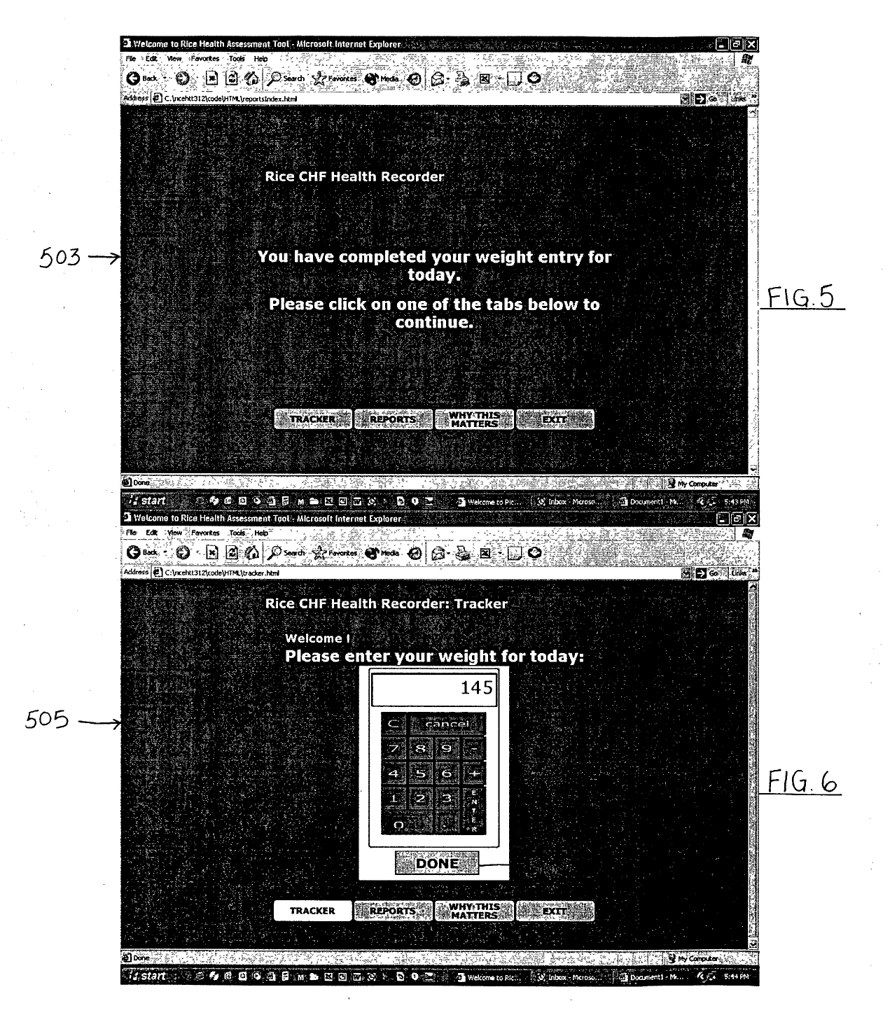

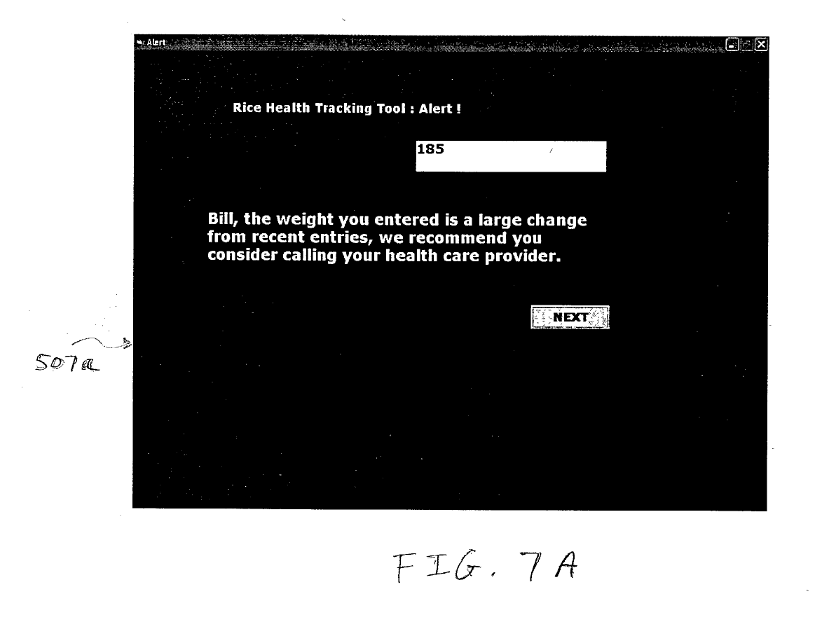

In FIG. 5, the next exemplary screen shot 503 confirms that the user has completed the weight entry for the day and prompts the user to click on the appropriate tab to continue. The user has several options, for example, the user may choose to get a report by pressing the “REPORTS” button 506, get information on “WHY THIS MATTERS” by pressing button 508, or exit the system by pressing the “EXIT” button 510.

FIG. 6 illustrates the next exemplary screen shot 505, which indicates that the patient entered a weight of 145 the next time. Once the patient enters the weight, the button 504 labeled “Done” is pressed to continue. It should be noted that in other embodiments, the patient may be asked to confirm the entry.

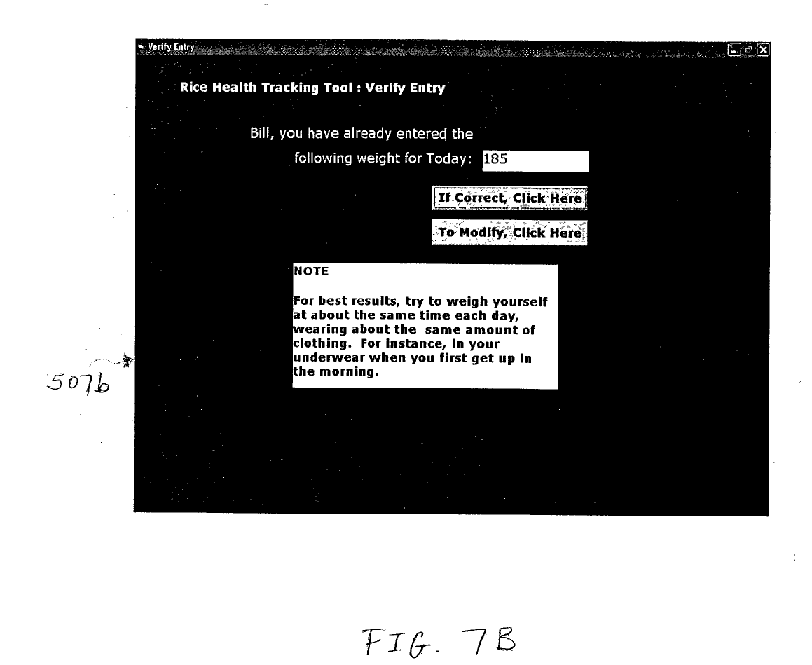

FIG. 7A and 7B illustrate the next exemplary screen shots 507a and 507b. In FIG. 7A, the body weight entered of 185 exceeds a control range for the particular patient, causing the system to give the patient an “Alert” stating, for example, that “the weight you entered is a large change from recent entries, we recommend you consider calling your healthcare provider.”

Furthermore, because the weight of 185 is entered after the initial entry of 125 on the same day, FIG. 7B shows a subsequent screen shot with a message stating, for example, that “you have already entered the following weight for today,” and a message stating, for example, that “For best results, try to weigh yourself at about the same time each day, wearing about the same amount of clothing.”

The system collects data, which undergoes a statistical analysis using an averaging program and self-comparison of data. The system uses a control range established by, for example, the Deming statistical method. In an example, when the weight of the patient exceeds about 3% of the control range, the system will produce an Alert to the patient. Statistical analyses steps for congestive heart failure would be as follows: (1) establish a base line weight associated with an initial stable condition for the patient; (2) perform an analysis under consistent guidelines to establish weight data going forward; (3) record the weight data and compare the data to the baseline to determine a percentage weight change from the base line; (4) if the percentage weight change represents a weight greater than a set percentage for the patient, generate an alert.

Statistical analysis for other chronic diseases would be approached in a similar manner; that is, a baseline for one or more parameters would be set; subsequent data would be collected from the patient for those parameters; and statistical changes for the parameters would be established based in part upon the character of the disease process and the particular details of the patient. The statistical changes that are used to analyze the patient will be dependent upon the character of the disease, the volatility inherent in the data being measured and other factors.

Referring now to FIG. 8, an exemplary screen shot 509, which illustrates a graphical report, is presented. As discussed above, the user may choose to obtain a report by simply pressing the “REPORTS” button 506. The report shows tracking of the parameter associated with the patient's clinical status. In this example, a graph of the measured body weight over a specified period of time is shown. It should be noted that the patient may choose the period of time reported, such as ten days, or thirty days, or another time interval.

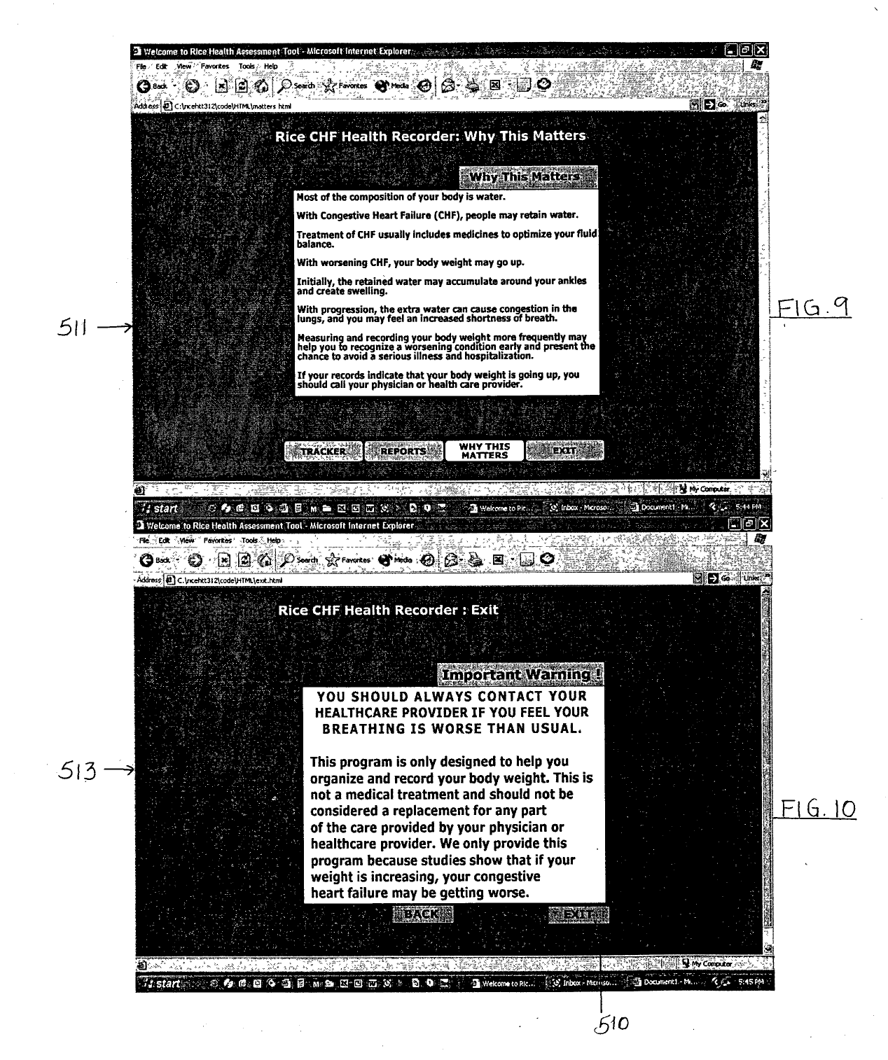

Referring now to FIG. 9, a screen shot 511, which explains the importance of tracking a parameter, is presented. The user may choose to get more information on the significance of the tracking of the parameters by simply pressing the “WHY THIS MATTERS” button 508. Exemplary screen shot 511 explains the importance of tracking weight in CHF patients and prompts the patient to call a physician or healthcare provider if the records indicate that his or her body weight is increasing.

CHF is a chronic disease characterized by a heart muscle that cannot pump blood effectively. Patients with CHF generally have difficulty breathing because excess fluids “behind” a weakened heart accumulate in the lungs. Care for CHF patients includes medicines such as diuretics to improve breathing by removing excess fluids. With the removal of excess fluids, the patient's lungs become “clear,” which allows the patient to breathe normally. Because water is the primary component of the human body, body weight measurements (on an ongoing basis) are an excellent indicator of the clinical status of a patient with CHF. The current care of most CHF patients includes visits to physicians' offices approximately every 3 to 6 months, depending on the severity of symptoms. By monitoring body weight twice a week, hospitalization rate and corresponding costs can be reduced by approximately 50-90%. Repetitive clinical monitoring of body weight, for example, twice a week, in CHF patients should be the “standard of care.”

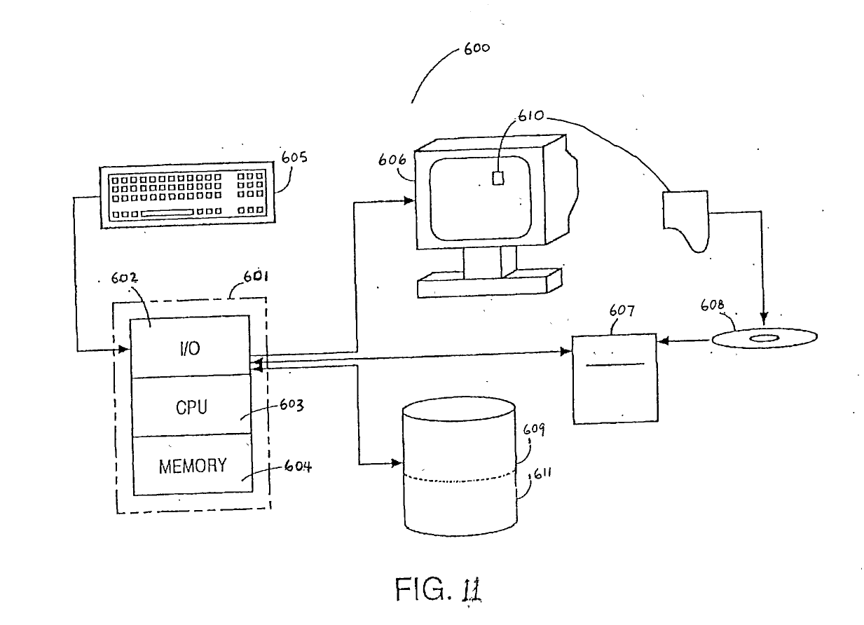

FIG. 10 illustrates an exemplary screen shot 513 that appears when the user desires to exit the system. The user exits the system by selecting the “EXIT” button 510. A disclaimer or warning to the patient may be displayed as a result.

In an embodiment, information that is taken remotely from a patient is analyzed statistically for that patient, and a determination is made as to whether or not that information may be indicative of a worsening medical condition that may require intervention by a healthcare professional, rather than attempting to treat the potentially worsening medical condition of the patient from a remote location. In this embodiment, instead of treating the medical condition of the patient from a remote location by using computers and the internet with conventional schemes, the patient would simply be notified of the fact that there may be cause for additional review or that the patient should seek medical intervention by a healthcare professional based upon the results of statistical analysis of one or more pre-selected parameters associated with a chronic disease. As a result of this notification to the patient, the patient may actually be encouraged to visit a physician's office, rather than attempting to avoid office visits.

For many chronic diseases, the worsening of a patient's condition may not follow a predictive model, and standardized therapies based upon broad demographic models are not suitable, thereby making it difficult to treat some types of chronic diseases remotely using standardized therapies. In general, certain parameters are associated with certain types of chronic diseases. For example, a patient's weight is generally associated with congestive heart failure, whereas peak flow is generally associated with asthma. Glucose is generally associated with diabetes, whereas mood and depression charts are generally associated with mental health problems.

In an embodiment, statistical models that have been applied to chaotic systems, such as to weather forecasting by NOAA, are applied to one or more preselected parameters of the patient associated with a chronic disease to determine the probability of worsening medical condition of the patient. By alerting the patient of the potentially worsening medical condition, the disease may be diagnosed, treated or managed early on by a healthcare professional, thereby avoiding more catastrophic and costly medical intervention later.

The methods and apparatus of the present invention, or certain aspects or portions thereof, may take the form of program code (i.e., instructions) embodied in tangible media, such as floppy diskettes, CD-ROMS, hard drives, or any other machine-readable storage medium, wherein, when the program code is loaded into and executed by a machine, such as a computer, the machine becomes an apparatus for practicing the invention. The methods and apparatus of the present invention may also be embodied in the form of program code that is transmitted over some transmission medium, such as over electrical wiring or cabling, through fiber optics, or via any other form of transmission, wherein, when the program code is received and loaded into and executed by a machine, such as a computer, the machine becomes an apparatus for practicing the invention. When implemented on a general-purpose processor, the program code combines with the processor to provide a unique apparatus that operates analogously to specific logic circuits.

FIG. 11 illustrates a typical computer system. While the figure illustrates traditional components of a personal computer, the present invention can have components similar to those shown, and furthermore, through accessing the Internet, the system may interact and interface with components on larger computers similar to examples illustrated in the figure.

A general purpose workstation computer 600 comprises a processor 601 having an input/output (“I/O”) section 602, a central processing unit (“CPU”) 603 and a memory section 604. The I/O section 602 is connected to a keyboard 605, a display unit 606, a disk storage unit 609 and a CD-ROM drive unit 607. The CD-ROM unit 607 can read a CD-ROM medium 608 that typically contains programs and data 610. The disk storage unit can be, or is connected to, a database or network server 611. The connection can be via a modem or other digital communication devices, such as wireless receiver and transmission components as used in PDAs and wireless communication devices known to one of ordinary skill in the art. The database server and network server 611 can be the same device or two separate but coupled devices.

The computer 600 may be a network appliance, personal computer, desktop computer, laptop computer, set top box, web access device (such as WEBTV® (Microsoft Corporation)), or the like. Use of computers also contemplates other devices similar to or incorporating computers, such as personal computers, television interfaces, kiosks, and the like.

Embodiments of the present invention may be implemented in a standalone system, entirely on the patient's computer hard drive so that there are no privacy or security concerns. The method according to embodiments of the present invention does not necessarily need a computer at all. A person could use a telephone, a personal digital assistant (PDA), or other means to record the data measurements described above. The patient also could be alerted by telephone, PDA, or such other means.

From the above description of the invention it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are to be considered in all respects as illustrative and not restrictive. It should also be understood that the invention is not limited to the particular embodiments described herein, but is capable of many equivalents, rearrangements, modifications, and substitutions without departing from the scope of the invention.

What is claimed is:

1. A method for notifying a patient to contact a healthcare professional, comprising the steps of:
performing repetitive interval evaluations of one or more pre-selected disease-associated parameters of the patient to obtain a series of evaluation results;
performing a statistical analysis of the evaluation results;
determining whether the statistical analysis indicates that the evaluation results fall outside a desired range; and
notifying the patient if the evaluation results fall outside the desired range.

2. The method of claim 1, wherein the step of notifying the patient comprises the step of advising the patient to contact a healthcare professional.

3. The method of claim 1, wherein the step of performing the statistical analysis comprises the step of comparing most recently measured data for said one or more pre-selected disease-associated parameters with prior historical data for said one or more pre-selected disease-associated parameters.

4. The method of claim 1, wherein said one or more pre-selected disease-associated parameters include a body weight of the patient associated with congestive heart failure.

5. The method of claim 1, wherein said one or more pre-selected disease-associated parameters include a peak flow associated with a disease selected from the group consisting of asthma and emphysema.

6. The method of claim 1, wherein said one or more pre-selected disease-associated parameters include glucose associated with diabetes.

7. The method of claim 1, wherein said one or more pre-selected disease-associated parameters include mood and depression charts associated with a mental health disease.

8. The method of claim 1, wherein said one or more pre-selected disease-associated parameters include those associated with a cardiovascular disease.

9. The method of claim 1, wherein said one or more pre-selected disease-associated parameters include those associated with rehabilitation from surgical interventions.

10. The method of claim 1, wherein said one or more pre-selected disease-associated parameters include those associated with cancer therapies.

11. The method of claim 1, wherein said one or more pre-selected disease-associated parameters include those associated with cancer.

12. A computer program product including a medium readable by a processor, said medium having stored thereon a sequence of instructions which, when executed by said processor, causes said processor to execute a process for optimizing early detection of an exacerbating medical condition of a patient, said process comprising the steps of:
reading repeatedly measured one or more disease-associated parameters;
performing a statistical analysis of progression of said one or more disease-associated parameters; and

13. notifying the patient if the statistical analysis of said one or more disease-associated parameters is indicative of an exacerbating medical condition.

14. The computer program product of claim 12, wherein the step of performing the statistical analysis comprises the step of comparing most recently measured data for said one or more disease-associated parameters with prior historical data for said one or more disease-associated parameters.

15. The computer program product of claim 12, wherein the step of notifying the patient comprises the step of advising the patient to contact a healthcare professional.

16. A system comprising a computer having a processor to execute a program for notifying a patient of changing disease-associated parameters indicative of an exacerbating medical condition, the program including instructions which, when executed by the processor, causes the computer to perform the steps of:
initializing a downloaded applet into the computer;
allowing access to the patient;
receiving disease-associated parameter input data;
performing a statistical analysis of the disease-associated parameter input data;
providing an advisory statement if the statistical analysis is indicative of an exacerbating medical condition; and
exiting the program.

17. The system of claim 15, further comprising the step of outputting a report of the disease-associated parameter input data.

18. The system of claim 16, wherein the step of outputting the report comprises the step of outputting a graph for a particular time-frame.

19. The system of claim 15, wherein the computer is standalone.

20. The system of claim 15, wherein the step of initializing comprises the step of entering a name of the patient.

21. The system of claim 15, further comprising the step of downloading the applet again for use by another patient.

22. The system of claim 15, wherein the step of allowing access to the patient comprises the step of double clicking a desktop icon on the computer.

23. The system of claim 15, wherein the step of receiving the disease-associated parameter input data comprises the step of recording data including a body of weight of the patient.

24. The system of claim 15, wherein the step of receiving the disease-associated parameter input data comprises the step of recording data including a peak flow.

25. The system of claim 15, wherein the step of receiving the disease-associated parameter input data comprises the step of recording data including glucose.

26. The system of claim 15, wherein the step of receiving the disease-associated parameter input data comprises the step of recording data including mood and depression charts.

27. The system of claim 15, wherein the step of receiving the disease-associated parameter input data comprises the step of receiving inputs by clicking on a number pad by the patient.

28. The system of claim 15, further comprising the step of confirming reception of the disease-associated parameter input data.
